(19) United States
(12) Patent Application Publication
(10) Pub. No.: US 2007/0270673 A1
(43) Pub. Date: Nov. 22, 2007

(54) OCULAR PARAMETER SENSING FOR CEREBRAL PERFUSION MONITORING AND OTHER APPLICATIONS

(76) Inventors: Daniel J. Abrams, Denver, CO (US); Christopher Crowley, Golden, CO (US); Kevin Keilbach, Boulder, CO (US)

Correspondence Address: MARSH, FISCHMANN & BREYFOGLE LLP, 3151 SOUTH VAUGHN WAY, SUITE 411, AURORA, CO, 80014, US

(21) Appl. No.: 11/675,178

(22) Filed: Feb. 15, 2007

Related U.S. Application Data
(63) Continuation-in-part of application No. 11/567,597, filed on Dec. 6, 2006.
(60) Provisional application No. 60/748,101, filed on Dec. 6, 2005.

(51) Int. Cl. A61B 5/00

(52) U.S. Cl. 600/310

(57) ABSTRACT

A system and associated methodology allow for monitoring ocular parameters of a patient. Parameters that may be monitored in this regard include ocular perfusion, retinal oxygen saturation and ocular pressure. In one implementation, a device for monitoring the desired parameters includes a contact lens with fiber optic pathways mounted thereon. Light of multiple wavelengths can be transmitted into the patient's eyes via input optical pathways. Output optical pathways are associated with a camera for obtaining images of an area of interest within the patient's eyes. The images can be processed to obtain information regarding ocular perfusion and/or oxygen saturation. Changes in this regard can be used to identify a condition of interest.

### CROSS-REFERENCE TO RELATED APPLICATION

This application is a Continuation-In-Part application that claims priority from U.S. application Ser. No. 11/567,597, entitled, “Intra-Operative Ocular Parameter Sensing,” filed Dec. 6, 2006, which claims priority from U.S. Provisional Application No. 60/748,101, entitled: “Intra-Operative Visual Pressure and Perfusion Change Detector,” filed on Dec. 6, 2005, the contents of both of which are incorporated herein as if set forth in full.

### FIELD OF INVENTION

The present invention relates generally to measuring ocular parameters, e.g., related to ocular perfusion and/or pressure. The invention can be used to monitor cerebral perfusion, central circulatory flows or eye health, or to detect the onset of various conditions, including certain post-operative complications.

### BACKGROUND OF THE INVENTION

Ocular parameters, such as parameters related to ocular perfusion (e.g., oxygen saturation of the retina or fundus), may be monitored for a variety of reasons. Ocular perfusion provides a direct indication of eye health and may therefore be monitored to identify potential eye disease or deterioration. For example, ocular perfusion may be monitored to detect hypoxia of the retina or ONH, which has been linked to a variety of ocular vascular disorders. However, ocular perfusion may also be monitored, for example, as an indication of cerebral perfusion, central circulatory flows or to identify potential surgical complications.

With respect to cerebral perfusion, it has been recognized that retinal oxygen saturation is well correlated to cerebral blood flow. There are a variety of contexts in which it may be desired to monitor cerebral blood flow or, more particularly, to monitor the perfusion and oxygenation of each side of the brain relative to systemic perfusion. For example, monitoring cerebral blood flow is of direct interest in connection with a variety of conditions such as Closed-Head Injuries, cerebral aneurysm, strokes (DVAs) or other conditions involving loss of neuronal function due to disturbance in cerebral perfusion. Cerebral perfusion may also be monitored in connection with a variety of medical conditions/procedures, including: heart surgery (e.g., CABG, ablation for treatment of atrial fibrillation, aortic valve surgery) and coronary grafts, carotids, neck surgeries, aneurysm surgeries, anything done in the upright or sitting position, cerebrovascular disease, cognitive deficits, etc. In addition, because central circulatory flows, including to the retina, are preserved in certain medical conditions where peripheral flows may be compromised, monitoring ocular perfusion and oxygen saturation may be useful to supplement or supplant conventional finger or other extremity oxygen saturation measurements in appropriate cases.

Identifying potential post-operative complications, including post-operative visual loss (POVL), is also a significant concern. Surgical complications exact a considerable toll on patient health and quality of life, in addition to increasing healthcare costs by extending treatment, requiring longer hospital stays, and creating economic and personal hardship due to death and disability. A 2003 study published in the Journal of the American Medical Association reviewed postoperative complications and 18 types of medical injuries during hospitalization. The study concluded that the events accounted for 2.4 million additional hospital days and $9.3 billion in excess charges each year. In the same year of study's publication, the Centers for Medicare & Medicaid Services (CMS) and the Centers for Disease Control and Prevention (CDC) formed the Surgical Care Improvement Project (SCIP) with ten other national organizations. In 2005, they announced an ambitious goal of improving the safety of surgical care by reducing post-operative complications 25% by 2010.

Because the large numbers of spinal operations are increasing each year and the operations themselves are increasing in complexity, it is critical to minimize surgical complications from back operations. Over 320,000 back operations are performed each year in the United States, including discs, lumbar laminectomies and fusions of lumbar and thoracic spine. These procedures are expected to increase as the population ages. As much as 80% of the U.S. population will be affected by back pain at some time during their lives. According to injury statistics from the US Bureau of Labor Statistics, there were 303,750 work-related back injuries in 2003. The aging process itself can lead to back pain that may require treatment and surgery, and because people today are living longer, the number of patients with back pain increases every year. Members of the Baby Boom generation will be the largest population of older adults in history who may require joint and back treatment or surgery.

Anesthesiologists have led the way in improving surgical outcomes in the United States through early detection of parameters that lead to adverse outcomes. In July 1999, The American Society of Anesthesiology (ASA) Committee on Professional Liability established the ASA POVL registry, which recognized POVL as a significant concern with an apparent increase in incidence. One possibility for the apparent increase in incidence in blindness, in spite of the absence of concrete incidence data, is related to an increase in longer spine surgeries occurring in the last ten years. Malpractice awards for POVL are often over $250,000.

Criteria for POVL include an acute deficit in vision not attributable to other causes for visual loss, which is usually noticed immediately after surgery. The blindness may be bilateral (50%) and in some cases partially reversible (44%). Patients may have complete blindness; partial blindness with diminished visual acuity, field deficits and loss of color vision. This complication probably relates to optic nerve ischemia (caused by visual system arterial insufficiency or visual system venous hypertension) but also may be a result of direct pressure on the eye during surgery. There is currently no accepted and effective mechanism for preventing this complication. As discussed below, it has been recognized that monitoring ocular parameters, including ocular perfusion/saturation and/or pressure, provides a mechanism for detecting the onset of such complications.

### SUMMARY OF THE INVENTION

The present invention is directed to devices and associated methodology for monitoring one or more ocular parameters such as ocular perfusion, oxygen saturation and/or pressure. As noted above, these parameters may be monitored in relation to analyzing eye health, central circulatory flows, cerebral conditions or other conditions. Moreover, the parameters may be monitored in relation to a medical procedure (e.g., before, during and/or after) or independent of any such procedure. For example, the invention can be used to monitor a patient during a medical procedure to identify a condition indicative of the possible onset of blindness or damage to the patient's eye or eyes. This provides an opportunity for the caring physicians to address the condition, e.g., by repositioning the patient or otherwise attempting to increase perfusion and oxygen saturation of the retina or fundus, thereby potentially reducing the occurrence or severity of such complications.

The structure and methodology of the invention may also be used to monitor eye health and patient health in other contexts. For example, as noted above, because ocular perfusion is a good indication of central circulatory flows, ocular perfusion may be monitored in lieu of or in addition to extremity measurement in certain cases. Relatedly, because retinal oxygen saturation is well correlated to cerebral blood flow, measurements performed on the patient's eye in accordance with the present invention may be useful in noninvasively monitoring cerebral conditions with greater confidence. Moreover, a variety of conditions related to eye diseases or deterioration may be identified or analyzed using the devices and processes described herein. In addition, the processes discussed below may be used in conjunction with other technologies, including certain existing ophthalmology technologies, for improved patient monitoring and diagnosis. For example, the processes described below may be useful together with fluorescent angiography to determine if there has been damage to one of the layers of the retina.

Some interesting background discussion and related technology is discussed in: de Kock, et al., Reflectance Pulse Oximetry Measurements from the Retinal Fundus, IEEE Transactions on Biomedical Engineering, Vol. 40, No. 8, August 1993; and Khoobehi, et al., Hyperspectral Imaging for Measurement of Oxygen Saturation in the Optic Nerve Head, Investigative Ophthalmology & Visual Science, Vol. 45, No. 5, May 2004. In particular, these articles discuss various methodologies for analyzing ocular perfusion and oxygen saturation and certain structure for implementing those methodologies. However, those articles are not directed to post-procedure blindness and do not utilize fiber optic technology as set forth herein.

In accordance with one aspect of the present invention, a method and apparatus (“utility”) is provided for monitoring ocular perfusion and/or oxygen saturation. As noted above, it may be desirable to monitor ocular perfusion or oxygen saturation in connection with certain medical procedures. In addition, there may be other circumstances where it is desired to monitor ocular perfusion or oxygen saturation, e.g., after eye surgery or otherwise to monitor eye health. The utility involves an optical instrument for use in monitoring patient perfusion and/or oxygen saturation and a positioning structure for positioning at least a portion of the optical instrument so as to monitor the noted ocular parameter(s). For example, the optical instrument may include an optical transmitter assembly for transmitting an optical signal in relation to tissue of a patient's eye and an optical receiver assembly for receiving the optical signal after interaction with the tissue, e.g., reflection from the tissue area. Such a device may further include a processor for providing information regarding perfusion or oxygen saturation. For example, perfusion or oxygen saturation may be monitored based on measurements of signal attenuation at one or more wavelengths. A variety of algorithms have been developed in this regard. The positioning structure may include an eye contact for maintaining the instrument portion in a substantially fixed position in relation to a retina of the patient. For example, the instrument portion may include a fiber end for transmitting or receiving the optical signal. Alternatively or additionally, the utility may include headgear, e.g., in the form of eye glasses or virtual realty-style headgear, for reducing ambient light interference and/or providing the noted positioning structure.

In accordance with another aspect of the present invention, a fiber optic pathway is used in performing imaging spectroscopy to determine oxygen saturation, e.g., of ocular or other tissue. An associated apparatus comprises an imaging system for obtaining at least one image of tissue of interest; at least one fiber optic pathway disposed between the imaging system and the tissue of interest for use in obtaining the image(s); and a processor for processing the images to obtain information regarding the oxygen saturation of the tissue of interest. The fiber optic pathway(s) may be used to transmit light to the tissue of interest and/or transmit reflected light from the tissue of interest to an optical receiver (e.g., a camera). The processor may be operative for digitally subtracting images corresponding to different wavelengths and to correlate the result to an oxygen saturation value. In this regard, the processing may effectively combine reflectance imaging and oximetry.

In accordance with one aspect of the present invention, a utility is provided for use in detecting a potential onset of patient blindness. The utility involves identifying a physiological parameter potentially related to an onset of patient blindness, monitoring the identified physiological parameter during a medical procedure, and, based on monitoring, taking potentially corrective action in the event of an indication related to a potential onset of patient blindness. It will be appreciated that any physiological parameter determined to be relevant to the onset of patient blindness may be monitored in this regard. Parameters that may be relevant in this regard include ocular pressure, perfusion and oxygen saturation. Any one or combination of these parameters may be monitored during the medical procedure. With regard to perfusion and oxygen saturation, the measurement may be in the arterial, capillary or venous phase of blood perfusion of the retina. These measurements can be perfusion or saturation related and will generally involve measurements of different wavelengths. It may be desirable to monitor such a parameter during a variety of medical procedures, including emergency room procedures and surgical procedures, among others. For example, post-operative blindness has been identified as a problem in relation to certain spinal procedures.

In accordance with a still further aspect of the present invention, a utility is provided for use in monitoring ocular pressure during a medical procedure. The utility includes an eye contact; a pressure sensor, supportably associated with the eye contact, for sensing an ocular pressure of the patient; and a monitoring instrument, supportably associated with the pressure sensor, for use in monitoring the ocular pressure of the patient during a surgical procedure and for providing an indication upon detecting a condition potentially related to an onset of blindness. For example, the monitoring instrument may include a MEMS tonometer or the like. The sensor may be supported on an eye contact, for example, in the form of a contact lens. The pressure parameter may be monitored together with ocular perfusion or oxygen saturation to identify a condition of interest. The condition of interest may be identified based on any one of these parameters or any combination thereof.

### BRIEF DESCRIPTION OF THE DRAWINGS

For a more complete understanding of the present invention and further advantages thereof, reference is now made to the following detailed description taken in conjunction with the drawings in which:

FIG. 1 is a schematic diagram of a patient monitoring system in accordance with the present invention;

FIG. 2 is a schematic diagram of an embodiment of a patient monitoring system in accordance with the present invention;

FIGS. 3A-3C show various implementation details of a retinal oxygen saturation monitoring system in accordance with the present invention;

FIG. 4 shows details of an ocular pressure and oxygen saturation monitoring system in accordance with the present invention;

FIG. 5 is a flow chart illustrating a patient monitoring process in accordance with the present invention; and

FIGS. 6A-6E show various views of an optical system in accordance with the present invention.

### DETAILED DESCRIPTION

In the following description, the invention is set forth in the context of a patient monitoring system for monitoring certain ocular parameters of a patient. These parameters may be monitored in a variety of contests, including during a medical procedure. Such a system can be used to address the objective of identifying conditions indicative of potential post-procedure blindness so that these conditions can be addressed by the caring physicians. However, it will be appreciated that various aspects of the invention are applicable in other contexts. For example, the ocular parameters may be monitored independent of any separate medical procedure to monitor the patient's eye health or general health. Accordingly, the invention is not limited to the specific embodiments, implementations and contexts described below.

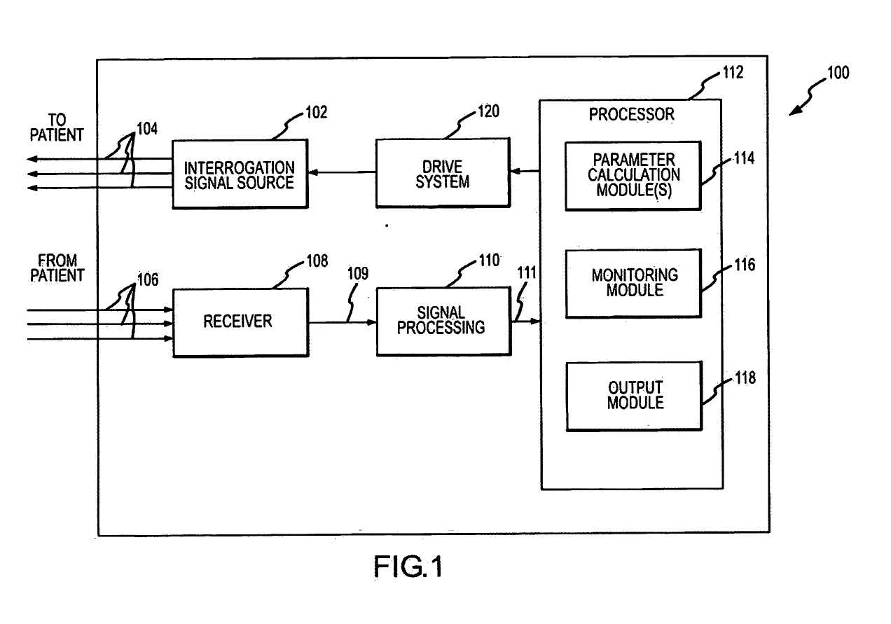

Referring to FIG. 1, a patient monitoring system 100 is shown. The patient monitoring system 100 is used to monitor certain ocular parameters such as ocular perfusion, retinal oxygen saturation or ocular pressure. As noted above, blindness may occur in connection with various medical procedures. For example, post-operative blindness is a particular concern in connection with spinal surgery. The monitoring system 100 can be used to monitor ocular parameters during such a medical procedure to identify parameter values or changes in parameter values that indicate the potential onset of problems in this regard. Accordingly, the system 100 may be disposed in the operating theater for use during such a procedure. The system 100 can be an independent system or may be incorporated into a larger system such as a multi-parameter monitoring system. Similarly, an output from the monitoring system 100 may be provided to a display or other output device used to monitor other parameters for the convenience of the monitoring physician or other personnel. As noted above, various parameters may be monitored to identify the ocular conditions of interest. Some of these parameters may be monitored with active monitoring systems, e.g., involving the transmission of interrogation signals such as optical signals to the patient's eye, and other monitoring systems may be passive, e.g., involving detecting pressure or another parameter free from the use of such interrogation signals. The illustrated system 100 includes an interrogation signal source 102 for transmitting interrogation signals 104 to the patient. The interrogation signal source 102 is controlled by a drive system 120 that, in turn, is controlled by a processor 112. In this regard, the processor 112 may control the timing, modulation, multiplexing or other characteristics of the interrogation signals 104. It will be appreciated that the transmitted signal may be continuous over the exposure period or may be pulsed depending on the application. The drive system receives instructions from the processor and drives the source 102 so as to generate appropriate signals 104. The service cycles for the sources may be selected for enhanced patient safety. Thus, for example, the sources may be operated intermittently or occasionally (e.g., only 10 seconds per minute) and at minimal intensity in order to minimize light and heat exposure at the cornea and retina. It will be appreciated that the interrogation signal source 102 and drive system 120 may be omitted for implementations involving only passive monitoring.

The illustrated system 100 further includes a receiver 108. The receiver 108 receives monitoring signals 106 from the patient. The nature of the receiver 108 varies depending on the parameters being monitored and the nature of the monitoring signals 106. Thus, for example, in the case of optical signals used to monitor retinal perfusion or oxygen saturation, the monitoring signals 106 may be optical signals (e.g., transmitted to the detector system via fiber optics) or may be electrical signals representative of optical signals (e.g., in the event of an upstream photodetector). In the case of a system for monitoring ocular pressure, an electrical signal indicative of ocular pressure may be received at the detector 108. Moreover, the number of interrogation signals 104 transmitted to the patient may differ from the number of monitoring signals 106 received from the patient. For example, in the case of a hybrid system including active and passive parameter monitoring (e.g., active retinal oxygen saturation or ocular perfusion monitoring and passive ocular pressure monitoring), the monitoring signals 106 may include signals corresponding to the interrogation signals 104 as well as signals relating to the passive monitoring. The monitoring signals 106 may be optical, electrical or other signals and, in the case of electrical signals, may be analog or digital signals.

The receiver 108 is operative to provide an output electrical signal 109 to the signal processing unit 110. Thus, in the case of optical monitoring signals 106, the receiver 108 may include a photodetector for receiving the optical signals and providing an associated output signal 109. The signal processing unit 110 processes the signal 109 to provide a processor signal 111 for use by the processor 112. Thus, the signal processing unit 110 may perform a number of functions including signal amplification, digital-to-analog conversion, demodulating or de-multiplexing and other signal conditioning.

Different technologies can be utilized to determine retinal oxygen saturation, with corresponding differences in transmitted and received optical signals, transmitting and receiving optical components and processing components. For example, depending on the implementation, the signal 109 may convey, for example, information corresponding to a pixel based image of the ocular fundus (e.g., of retinal tissue) or may be used to derive an analog value. In the case of a pixel-based image, the photodetector may include a CCD or CMOS imaging photodetector. In the case of deriving an analog value, the photodetector may include a single detector unit for providing a current or voltage signal representative of the light incident thereon.

The processor 112 receives the processor signal 111 and processes the signal 111 so as to enable substantially real-time patient monitoring. The illustrated processor 112 includes a parameter calculation module(s) 114, a monitoring module 116 and an output module 118. The parameter calculation module 114 calculates at least one physiological parameter believed to relate to post-operative blindness in the illustrated embodiment. For example, the parameter may be related to ocular perfusion, retinal oxygen saturation or ocular pressure (or changes in derivative/related values of any of the above). Thus, in the case of ocular pressure, the input signal 111 may be analyzed to determine a value related to ocular pressure or change in ocular pressure. Similarly, in the cases of retinal oxygen saturation or perfusion, a value related to these parameters or changes in these parameters may be calculated.

An image subtraction algorithm may be used in regard to oxygen saturation or perfusion measurements. Generally, these algorithms involve obtaining a first image at a first wavelength of transmitted light, obtaining a second image at a second wavelength, and digitally subtracting one image from the other to obtain a differential image. The differential image includes information that is correlated to the perfusion and oxygen saturation of the tissue under examination. The images can be processed to identify the optic nerve or other location of interest (e.g., the macula) for performing the perfusion or oxygen saturation analysis. Processing techniques specific to retinal oxygen saturation determinations may be used in this regard. Relevant technology in this regard is disclosed in the following references, which are incorporated herein by reference: Denninghoff, et al., Retinal venous oxygen saturation and cardiac output during controlled hemorrhage and resuscitation, Journal of Applied Physiology 94: 891-896, 2003; de Kock, et al., Reflectance Pulse Oximetry Measurements from the Retinal Fundus, IEEE Transactions on Biomedical Engineering, Vol. 40, No. 8, August 1993; and Khoobehi, et al., Hyperspectral Imaging for Measurement of Oxygen Saturation in the Optic Nerve Head, Investigative Ophthalmology & Visual Science, Vol. 45, No. 5, May 2004.

The monitoring module 116 is operative to use the calculated physiological parameter to identify a condition of interest. For example, an ocular pressure value, ocular perfusion value or oxygen saturation value (or changes therein) may be compared to a threshold to identify the condition of interest. In this regard, an ocular pressure above a certain threshold or above a threshold for a certain period of time may indicate a condition of interest. Similarly, an increase in ocular pressure over a given time interval may indicate a condition of interest. With regard to oxygen saturation, an oxygen saturation value dropping below a threshold value (e.g., below 70%) or below a threshold value for a certain length of time may indicate a condition of interest. Alternatively, a change in oxygen saturation, e.g., a drop of more than 20% over a given time period, may indicate a condition of interest. In any of these cases, the threshold values may be determined on a patient-by-patient basis, for example, in relation to a baseline value determined for the patient prior to initiation of the surgical procedure. The specific values used to determine the existence of a condition of interest may be determined theoretically or empirically. The values may balance the need to timely identify a condition of interest versus the annoyance and potential danger of false positives. Moreover, the values may be adjustable to allow specific physicians or institutions to select thresholds as desired. The data may be monitored continuously or frequently during a procedure entailing a high risk to the patient of visual loss.

The illustrated processor 112 further includes an output module 118 for providing output information that may be monitored by a physician or other personnel. For example, the output module 118 may output information continuously or, alternatively, only in the event of a condition of interest. In the latter case, the output module may output audio, visual or other alarms in the case of a condition of interest. In the case of continuous output, the output module 118 may output parameter values, such as instantaneous pressure or oxygen saturation values or a plot of such values over time. Additionally, the output module 118 may output a waveform reflecting such values over time or other information in the case of retinal oxygen saturation implementations.

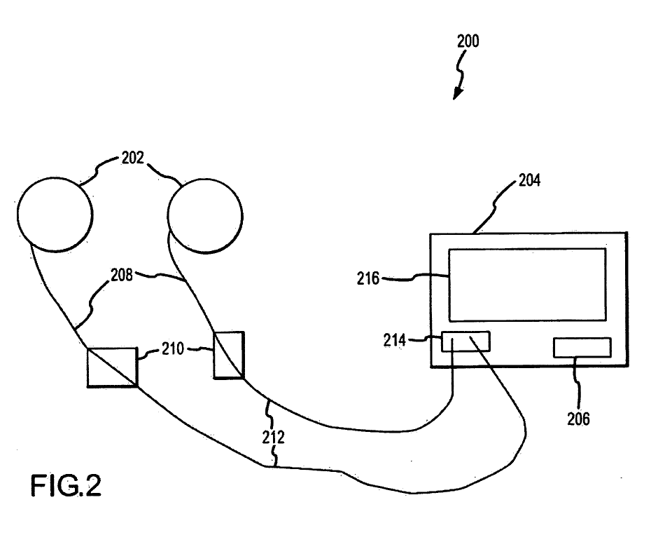

FIG. 2 illustrates a further embodiment of a monitoring system 200 in accordance with the present invention. For example, the system 200 may be used to monitor ocular pressure and/or retinal oxygen saturation during a surgical procedure. The illustrated system includes contact lens units 202 used to mount monitoring devices on the eyes of a patient during a surgical procedure, as will be described in more detail below. The contact lens units 202 are connected to a processing module 204 via contact connectors 208, instrument connectors 212, and cable connector boxes 210. The nature of the connectors 208 and 212 will vary depending on the nature of the parameter sensing system. Thus, for example, the connectors 208 and 212 may include optical fibers and/or electrical cables. The connector boxes 210 simplify the process of mounting the lenses on the patient's eye as this can be accomplished prior to connecting the contact connectors 108 to the connector boxes 210.

The connector boxes 210 may simply connect the contact connectors 208 to the instrument connectors 212. Alternatively, some processing may be implemented at the boxes 210. For example, the boxes 210 may include photodetectors such as cameras. In such a case, the connectors 208 may be fiber optic connectors whereas the connectors 212 may be electrical cables. The instrument connectors 212 connect to the processing module 204 at input ports 214. The connectors 212 may be detachably or permanently connected in this regard. The illustrated processing module further includes a display 216 and an on/off switch 206. The on/off switch allows the processing module 204 to be selectively enabled or disabled during medical procedures or otherwise as desired. The display 216 can provide parameter values and/or alarms or warnings in the case of conditions of interest.

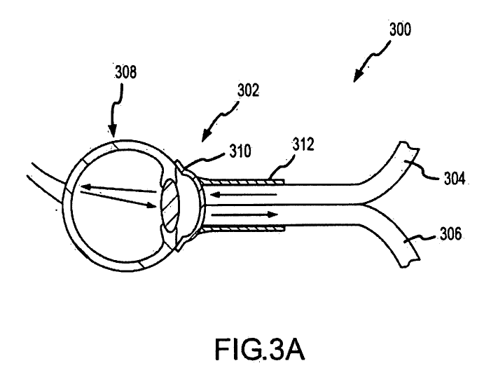

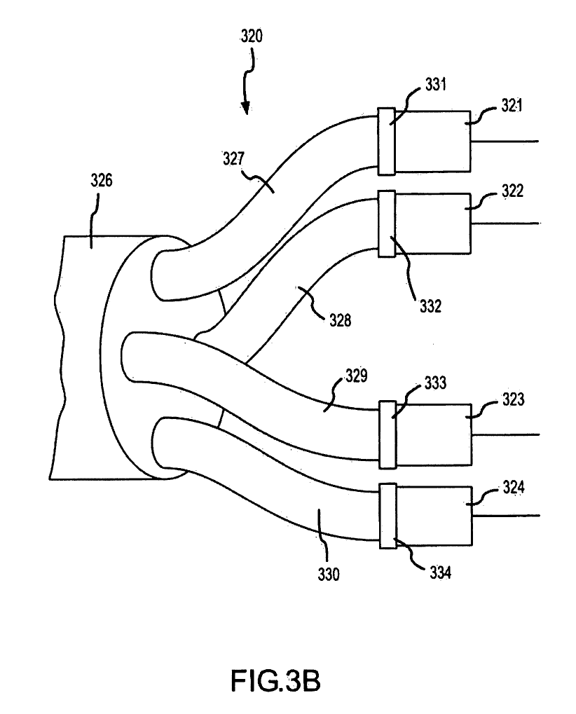

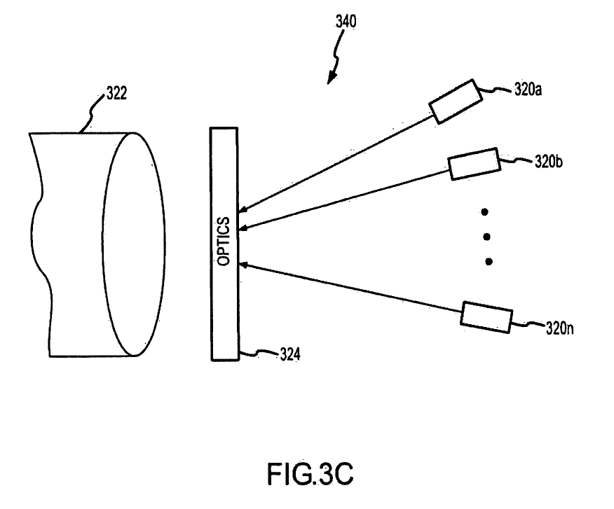

FIG. 3A illustrates a patient interface 300 that may be used to monitor ocular perfusion and/or retinal oxygen saturation. The interface 300 includes a mounting system for mounting input and output optical pathways 304 and 306 onto a patient's eye 308. The lens 310 may be placed onto the surface of the cornea at the beginning of a surgical procedure and may be replaced or moved as necessary during the course of the procedure. Moreover, the lens 310 may have channels for tear duct drainage and cooling of the eye. The lens 310 may be provided in different sizes to minimize damage to the cornea and is preferably sufficiently big or stable to minimize contact lens movement relative to the center of the pupil. In this manner, the relative positioning of the pupil and the optical pathways 304 and 306 remains relatively stable so that the amount of detected light will be sufficient for processing. If the amount of light received becomes insufficient, appropriate visual, audible or other alarms may be triggered to alert the caring physician(s) of the condition.

The mounting system 302 includes a contact lens 310 for placement on the patient's eye 308. Receptors 312 are mounted on the contact lens 310 and are dimensioned to receive the input and output optical pathways 304. The optical pathways 304 and 306 may be held in the receptors 312, for example, by a friction fit or by way of an adhesive or by way of a mechanical latching system or band that serves to trap and/or strain relieve the optical pathways 304 and 306. The ends of the optical pathways 304 and 306 may butt against the contact lens 310 or may be spaced therefrom. In this regard, an adhesive may be used to bond the ends of the optical pathways 304 and 306 to the surface of the contact lens 310. For optimal performance, the adhesive may be selected for transparency at the operating wavelengths and may be index matched to the optical pathways 304 and 306 and/or contact lens 310 for improved optical transmission. In addition, a clear gel may be used between the contact lens 310 and the patient's eye to cushion the eye and provide for enhanced optical transmission. The patient may be medicated or otherwise treated to inhibit eye movement during the procedure.

In operation, the input pathway 304 may be connected to one or more optical sources. For example, the sources may be operated to sequentially transmit light at multiple frequencies within the frequency range of 400-1000 nm so as to obtain multiple images. The output pathway 306 may be optically coupled to a CCD or CMOS camera or other optical detector. Although a single input pathway 304 and a single output pathway 306 are shown, it will be appreciated that multiple input and/or multiple output pathways may be used. As a further alternative, a single optical pathway may be used for the input and output optical signals. Moreover, each of the illustrated optical pathways may comprise a fiber optic bundle including numerous optical fibers. The bundle is preferably a coherent optical imaging bundle and is flexible and of low mass so as not to put excessive torque on the eye or contact lens.

As noted above, it may be desired to optically couple the input optical pathway to multiple optical sources, e.g., for providing light at multiple frequencies. The multiple sources may be coupled to the input optical pathway in a variety of ways. FIG. 3B illustrates a fiber optic coupling system 320. The system 320 is operative to couple multiple sources 321-324 to an input optical pathway 326. Although four sources 321-324 are shown, it will be appreciated that a different number of sources, including just one source or more than four sources, may be utilized. In the illustrated embodiment, the sources 321-324 are coupled to the input optical pathway 326 by fiber optic pigtails 327-330. The pigtails 327-330 are connected at one end to the input fiber optic pathway 326 and at the other end to sources 321-324 via mounting brackets 331-334. The mounting brackets 331-334 maintain the desired special relationship between the sources 321-324 and the pigtails 327-330 so that the light from the sources 321-324 is admitted into the ends of the pigtails 327-330. In this regard, the mounting brackets 331-334 may include optics or optical adhesives.

The other ends of the pigtails 327-330 are optically coupled to the input optical pathway 326. In the illustrated embodiment, this is accomplished by butt coupling the ends of the pigtails 327-330 to the end of the input optical pathway 326. An optical adhesive may be used to maintain this coupling. Alternatively, a bracket, together with optics or optical adhesives, may be used to couple the pigtails 327-330 to the input optical pathway 326.

Alternatively, the sources may be coupled to the input optical pathway by way of a free space interface. An embodiment of such a free space interface 340 is shown in FIG. 3C. The illustrated interface 340 is operative to optically couple sources 320a-320n to an input optical pathway 322. The sources 320a-320n are coupled to the pathway 322 via optics 324. For example, the optics 324 may comprise a lens for focusing light from the sources 320a-320n into the input optical pathway 322. Additionally or alternatively, the optics 324 may include a prism or diffraction grating for redirecting the light from the sources 320a-320n into the input optical pathway 322. In this regard, it is noted that the sources 320a-320n will generally operate at different wavelengths. Accordingly, a prism or diffraction grating allows light from the spatially separated sources 320a-320n to be redirected such that the light from the sources 320a-320n is directed axially into the pathway 322. Alternatively, a moveable mirror together with a lens or other optics may be used to selectively optically couple individual ones of the sources 320a-320n to the input optical pathway 322.

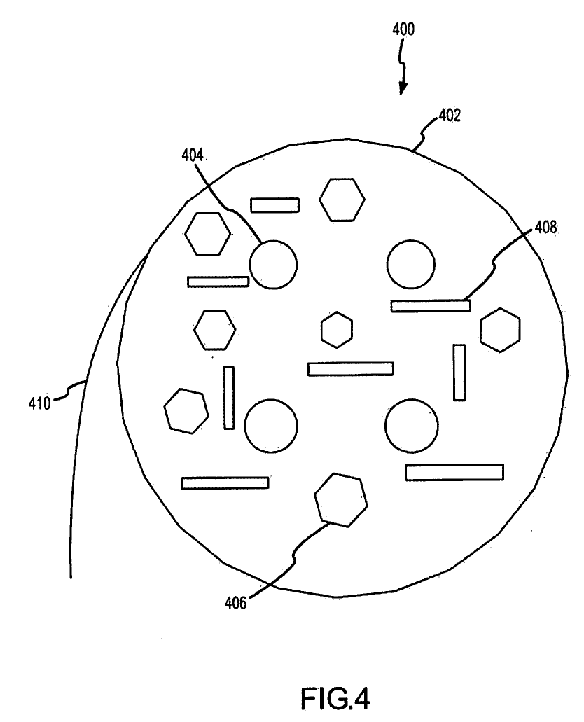

FIG. 4 illustrates an alternative patient monitoring system 400 in accordance with the present invention. The illustrated system 400 includes a contact lens 402 for placement on an eye of a patient. Mounted on the contact lens 402 are a number of optical emitters 404 (e.g., near infrared emitters), a number of optical detectors 406 (e.g., near infrared detectors) and a number of pressure sensors 408 (e.g., piezoelectric microrods). The contact lens 402 is connected to a processing module via an electrical connection 410 such as a beroptic and thin wire cable. The connector 410 is electrically coupled to the emitters 404 to control operation of the emitters to transmit optical signals into the eye of the patient. In addition, the connector 410 is electrically coupled to the detectors 406 and 408 to provide detector signals to the processing module.

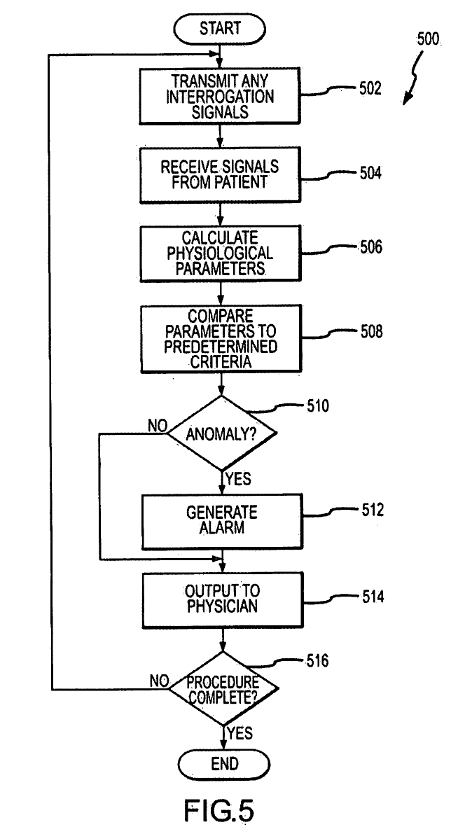

FIG. 5 is a flow chart summarizing a process 500 in accordance with the present invention. The process 500 is initiated by transmitting (502) any interrogation signals to the patient. As noted above, active or passive monitoring systems may be used. Accordingly, interrogation signals may or may not be necessary. In any event, monitoring signals are received (504) from the patient. For example, the signals may be optical signals, electrical signals representative of optical signals or pressure signals used to monitor the desired ocular parameters. The signals are processed to calculate (506) physiological parameters such as ocular perfusion, retinal oxygen saturation, ocular pressure or changes thereof. The parameters can then be compared (508) to predetermine criteria to identify a condition of interest such as a drop in retinal oxygen saturation or perfusion, or an increase in ocular pressure. If this comparison indicates an anomaly (510), then an alarm may be generated (512). For example, the alarm may be a visual, audio or other alarm. In cases of anomalies or in other cases, parameter information may be output (514) to the physician. Such output information may include oxygen saturation values, pressure values, plethysmographic waveforms or the like. This process can be continued (516) until a surgical procedure or other monitoring procedure is complete.

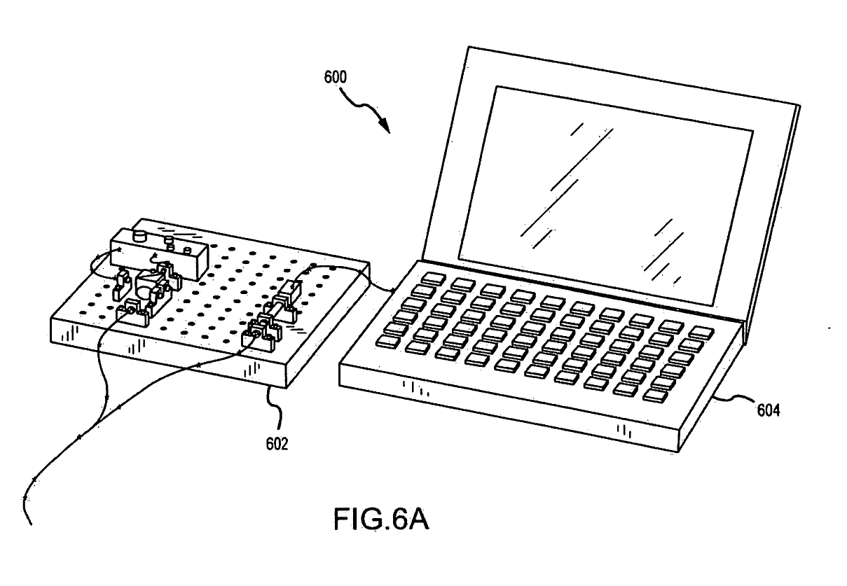

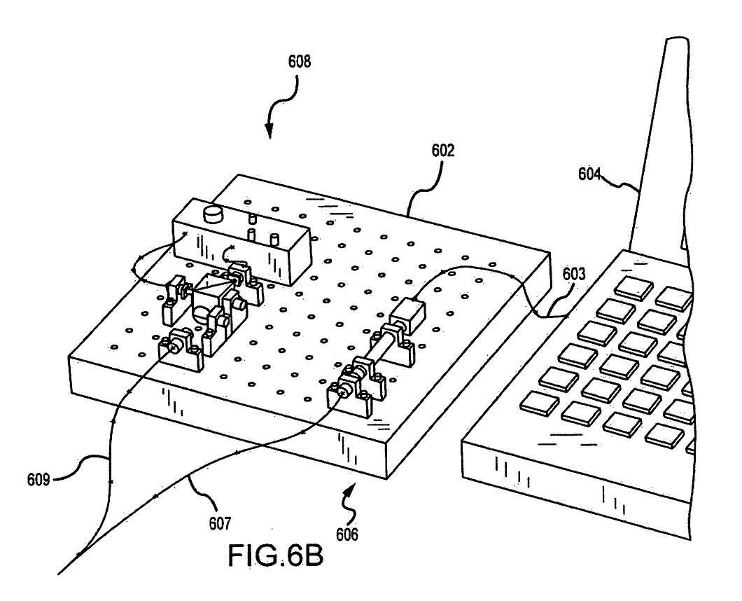

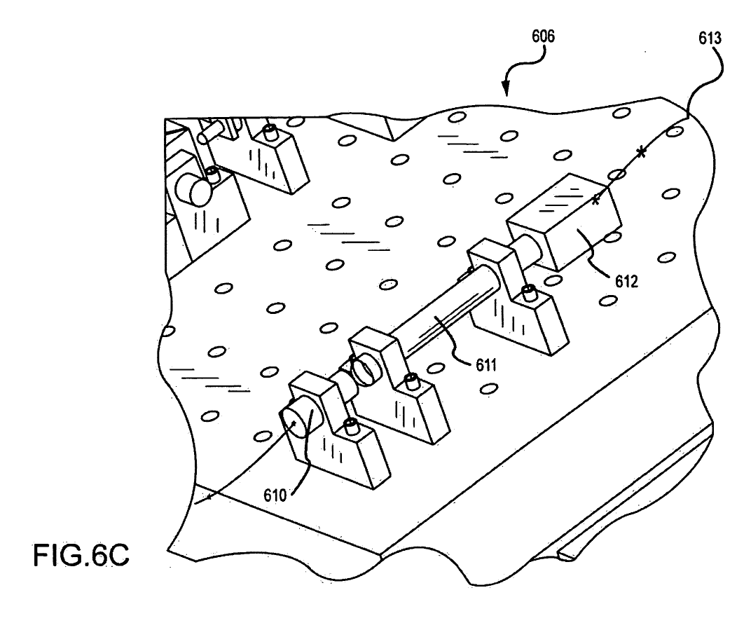

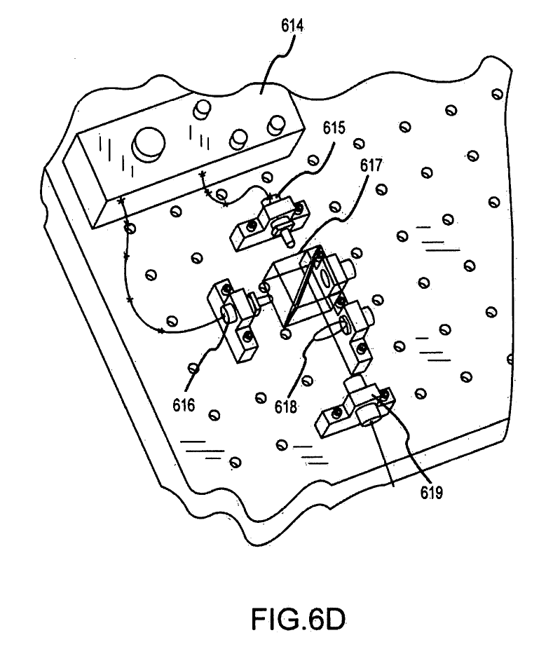

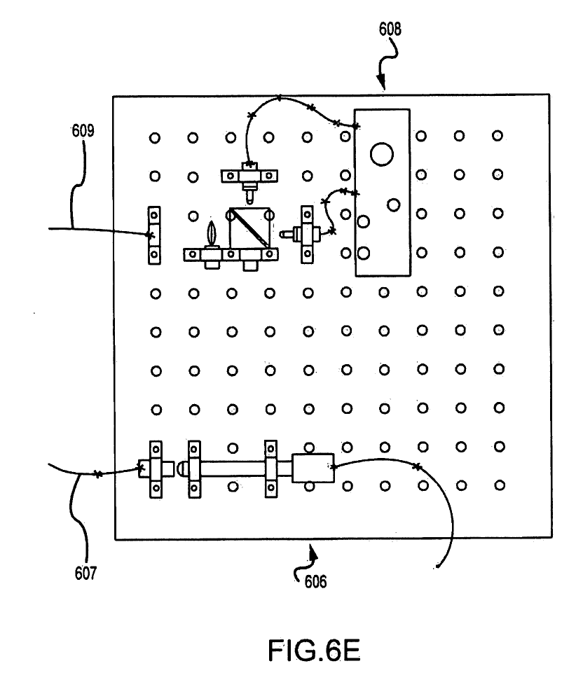

FIGS. 6A-6E illustrate an optical system 600 in accordance with the present invention. The illustrated system 600 includes an optical board 602, including a number of optical components as will be described below, and a processor 604 embodied as a computer. The optical board 602 is operative for transmitting optical beams of first and second wavelengths to a patient's eye via fiber optics and for receiving corresponding beams from the patient's eye as described above. The processor 604 is operative for calculating optical parameters as described above. It will be appreciated that the optical components of the board 602 and processing functionality of the processor 604 may be packaged in a single medical instrument housing. However, the illustrated embodiment of the system 600 allows for simple illustration of various functional components of the invention

The illustrated optical board 602 includes an optical transmission system 608 and an optical detection system 606. The optical transmission system 608 is operative to transmit optical signals into an input fiber 609 for transmission to one or both of the patient's eyes. As best shown in FIG. 6D, the optical transmission system 608 includes first and second optical sources 615 and 616 associated with drive components 614. For example, the first and second sources 615 and 616 may be LEDs for transmitting beams of first and second wavelengths. The drive components 614 may include electronics for driving the sources 615 and 616. In this regard, the drive components 614 may drive the sources 615 and 616 in response to signals from the processor 614 so as to provide the desired multiplexing or other timing of the transmitted signals.

In the illustrated embodiment, the optical beams from the sources 615 and 616 are both transmitted to the patient's eye via a common input fiber 609. This is accomplished in the illustrated embodiment by way of the dichroic beam combiner cube 617. The cube 617 is formed in two halves with an optical coating at the interface therebetween. The coating is substantially transparent to the beam transmitted by source 615 but is effective to redirect beams transmitted by the source 616 such that beams from the sources 615 and 616 exit the beam combiner 617 coaxially. The input fiber 609 terminates a ferrule that is mounted on the optical board 602. The beams exiting the beam combiner 617 are admitted into the input fiber 609 via optics 618, such as a focusing lens. The various components 614-619 are mounted on the optical board 602 by appropriate mounting mechanisms.

The optical detection system, as best shown in FIG. 6C, includes collection optics 611 and a detector 612. The output fiber terminates in a ferrule 610 mounted on the optical board 602. Light transmitted from the output fiber 607 is directed onto a detector surface of the detector 612 by the optics 612, which may include columnating or imaging lenses. The detector 612 receives the optical signal and generates an electrical signal corresponding to the optical signal. This electrical signal is transmitted to the processor 604 via cable 613. In this regard, the detector 612 may further include additional electronic components such as an analog-to-digital converter, an amplifier or other components for conditioning electrical signal from the detector 612.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

What is claimed:

1. An optical apparatus, comprising:
an eye contact; and
an optical fiber, supportably interconnected to said eye contact, for use in delivering or receiving an optical signal in relation to a patient's eye.

2. An apparatus as set forth in claim 1, further comprising an optical source for transmitting light to said patient's eye via said optical fiber.

3. An apparatus as set forth in claim 1, further comprising a photodetector for receiving light from said patient's eye via said optical fiber.

4. An apparatus as set forth in claim 3, wherein said photodetector comprises a camera for obtaining at least one image of a portion of said patient's eye.

5. An apparatus as set forth in claim 4, further comprising a processor for processing image information from said camera to determine an optical parameter value.

6. A patient monitoring device, comprising:
an optical instrument for use in monitoring one of patient perfusion and oxygen saturation, said optical instrument including at least one optical fiber for use in transmitting an optical signal for use in said monitoring; and
a positioning structure for positioning at least a portion of said optical instrument so as to monitor one of ocular oxygen saturation and ocular perfusion.

7. A device as set forth in claim 6, wherein said optical instrument includes an optical transmitter assembly for transmitting an optical signal in relation to tissue of a patient's eye and an optical receiver assembly for receiving said optical signal after interaction with said tissue.

8. A device as set forth in claim 7, further comprising a processor for providing information regarding said physiological parameter based on said received optical signal.

9. A device as set forth in claim 7, wherein said positioning structure comprises an eye contact for maintaining said instrument portion in a substantially fixed position in relation to a retina or cornea of said patient.

10. A device as set forth in claim 7, wherein said receiver assembly comprises an optical source, and eye contact for transmitting an optical signal relative to said patient's eye, and optical link for use in interconnecting said optical source to use in eye contact.

11. A device as set forth in claim 10, wherein said optical link includes an optical fiber.

12. A method for use in monitoring a patient, comprising the steps of:
identifying a physiological parameter potentially related to patient eye damage;
monitoring said identified physiological parameter during a medical procedure at a patient site separate from eyes of said patient; and
based on said monitoring, taking potentially corrective action in the event of an indication related to said onset of patient eye damage.

13. A method as set forth in claim 12, wherein said step of monitoring a physiological parameter involves monitoring an ocular parameter.

14. A method as set forth in claim 12, wherein said physiological parameter relates to ocular perfusion, oxygen saturation or pressure.

15. A method as set forth in claim 12, wherein said step of monitoring comprises identifying a change in ocular perfusion, oxygen saturation or pressure.

16. A method as set forth in claim 12, wherein said step of monitoring comprises operating a monitoring device to measure said physiological parameter during a spinal procedure.

17. A method as set forth in claim 12, wherein said step of monitoring comprises illuminating at least one eye of the patient with light of first and second wavelengths at first and second times, respectively.

18. A method as set forth in claim 17, wherein said step of monitoring further comprises obtaining first and second images of an area of interest of said eye corresponding to said first and second wavelengths.

19. A method as set forth in claim 18, wherein said step of monitoring further comprises obtaining differential values based on said first and second images.

20. A device for use in monitoring a patient, comprising:
an eye contact;
a pressure sensor, supportably associated with said eye contact, for sensing an ocular pressure of said patient; and
a monitoring instrument, supportable associated with said pressure sensor, for use in monitoring said ocular pressure of said patient during a medical procedure at a patient site separate from eyes of said patient and for providing an indication upon detecting a condition potentially related to an onset of eye damage.

21. A device as set forth in claim 20, further comprising an optical instrument for use in monitoring one of ocular perfusion and ocular oxygen saturation during said medical procedure.

22. A patient-monitoring apparatus, comprising:
an eye contact;
a pressure sensor, supportably associate with said eye contact, for monitoring an ocular pressure of said patient; and
an optical instrument, supportably associated with said eye contact, for use in monitoring an optical parameter in relation to said eye of said patient.

23. An apparatus as set forth in claim 22, further comprising a processor, operatively interconnected to said pressure sensor and said optical instrument, for identifying a condition of interest based on one or both of said ocular pressure and said optical parameter.

24. A method for use in monitoring a patient, comprising:
operating an optical instrument to monitor an ocular parameter, said optical instrument including an optical fiber for carrying an optical signal in relation to an eye of a patient; and
based on said monitored optical parameter, identifying a condition related to one of cerebral perfusion and central circulatory flow.

25. A method for use in monitoring a patient, comprising:
operating a first optical instrument to monitor a first ocular parameter related to perfusion;
operating a second instrument to monitor a second ocular parameter; and
monitoring a patient condition using said first and second parameters.
